(19) United States
(12) Patent Application Publication
(10) Pub. No.: US 2009/0286829 A1
(43) Pub. Date: Nov. 19, 2009

(54) QUINOLYNYLMETHYLIMIDIZOLES AS THERAPEUTIC AGENTS

(75) Inventors: Todd M. Heidelbaugh, Fountain Valley, CA (US); Phong X. Nguyen, Placentia, CA (US); Ken Chow, Newport Coast, CA (US); Michael E. Garst, Newport Beach, CA (US)

Correspondence Address: ALLERGAN, INC., 2525 DUPONT DRIVE, T2-7H, IRVINE, CA, 92612-1599, US

(73) Assignee: ALLERGAN, INC., Irvine, CA (US)

(21) Appl. No.: 12/436,837

(22) Filed: May 7, 2009

Related U.S. Application Data
(60) Provisional application No. 61/052,778, filed on May 13, 2008.

(51) Int. Cl. A61K 31/4709; A61P 25/00

(52) U.S. Cl. 514/314

(57) ABSTRACT

Disclosed herein are methods for treating a disorder associated with selective subtype modulation of alpha 2B and alpha 2C adrenergic receptors. Such methods can be performed, for example, by administering to a subject in need thereof a pharmaceutical composition containing a therapeutically effective amount of at least one compound having the structure:

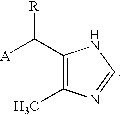

Compositions and medicaments related thereto are also disclosed.

### RELATED APPLICATION

This application claims the benefit of U.S. Provisional Application Ser. No. 61/052,778, filed May 13, 2008, the disclosure of which is hereby incorporated in its entirety herein by reference

### DESCRIPTION OF THE INVENTION

In one embodiment of the invention, disclosed herein are methods for treating a disorder associated with selective subtype modulation of alpha 2B and alpha 2C adrenergic receptors. Such methods can be performed, for example, by administering to a subject in need thereof a pharmaceutical composition containing a therapeutically effective amount of at least one compound having the structure:

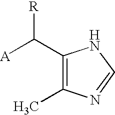

wherein R is H, C₁₋₄ alkyl, or CF₃;
A is quinolinyl having 0, 1, 2, or 3 stable substituents consisting of from 1 to 8 heavy atoms and any required hydrogen atoms, said heavy atoms being selected from C, N, O, S, F, Cl, Br, I, and any combination thereof.

Disorders that can be effectively treated by invention compounds possessing alpha 2B and alpha 2C selective subtype modulation activity include, but are not limited to, ocular disorders such as glaucoma, elevated intraocular pressure, optic neuropathy, corneal pain, diabetic retinopathy, retinal dystrophies, macular degeneration, non-exudative age related macular degeneration (ARMD), exudative Age Related Macular Degeneration (ARMD), Lebers optic neuropathy, optic neuritis often associated with multiple sclerosis, retinal vein occlusions, ischemic neuropathies and other neurodegenerative diseases, choroidal neovascularization, central serous chorioretinopathy, cystoid macular edema, diabetic macular edema, myopic retinal degeneration, acute multifocal placoid pigment epitheliopathy, Behcet's disease, birdshot retinochoroidopathy, intermediate uveitis (pars planitis), multifocal choroiditis, multiple evanescent white dot syndrome (MEWDS), ocular sarcoidosis, posterior scleritis, serpiginous choroiditis, subretinal fibrosis and uveitis syndrome, Vogt-Koyanagi-Harada syndrome, punctate inner choroidopathy, acute posterior multifocal placoid pigment epitheliopathy, acute retinal pigment epithelitis, acute macular neuroretinopathy, and following procedures such as photodynamic therapy and laser-assisted in situ keratomileusis (LASIK), and the like.

In other embodiments of the invention the disorders that can be effectively treated by invention compounds possessing alpha 2B and alpha 2C selective subtype modulation activity include chronic pain, visceral pain, neuropathic pain, cancer pain, post-operative pain, allodynic pain, neuropathic pain, causalgia, ischemic neuropathies, neurodegenerative diseases, diarrhea, nasal congestion, muscle spasticity, diuresis, withdrawal syndromes, neurodegenerative diseases, optic neuropathy, spinal ischemia, stroke, memory and cognition deficits, attention deficit disorder, psychoses, manic disorders, anxiety, depression, hypertension, congestive heart failure, cardiac ischemia, arthritis, spondylitis, gouty arthritis, osteoarthritis, juvenile arthritis, autoimmune diseases, lupus erythematosus, chronic gastrointestinal inflammations, Crohn's disease, gastritis, irritable bowel syndrome (IBS), functional dyspepsia, ulcerative colitis, allodynia, or a combination thereof.

In still other embodiments the disorders that can be effectively treated by invention compounds possessing alpha 2B and alpha 2C selective subtype modulation activity include central nervous system (CNS) motor disorders, such as L-dopa-induced dyskinesias, tardive dyskinesias, cervical dystonia, spinal torticollis, blepharospasm/Meige's disease, restless leg syndrome, essential tremor, rigidity (Parkinson's disease-associated or otherwise specified), ataxic disorder, spasticity, and the like.

In other embodiments of the invention, there are provided methods for treating a disorder associated with alpha 1A agonist activity. Such methods can be performed, for example, by administering to a subject in need thereof a pharmaceutical composition containing a therapeutically effective amount of at least one compound having the structure

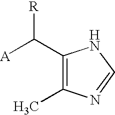

wherein R is H, C₁₋₄ alkyl, or CF₃;
A is quinolinyl having 0, 1, 2, or 3 stable substituents consisting of from 1 to 8 heavy atoms and any required hydrogen atoms, said heavy atoms being selected from C, N, O, S, F, Cl, Br, I, and any combination thereof.

Such disorders that can be effectively treated by compounds possessing alpha 1A agonist activity include, but are not limited to, stress urinary incontinence as well as other uses including dilation of the pupil, increase blood pressure, treating nasal congestion, and vasoconstriction in ocular tissue.

Treatment may be accomplished by administration orally, by injection, or any other means effective in delivering a therapeutically effective amount of the compound to the affected area. For example, the compound may be incorporated into a solid or liquid oral dosage form and administered regularly, such as once or twice a day, to the mammal or person.

### DEFINITIONS, EXPLANATIONS, AND EXAMPLES

Unless explicitly and unambiguously indicated otherwise, the definitions, explanations, and examples provided in this section shall be used to determine the meaning of a particular term or expression where there is any ambiguity arising from other parts of this document or from any disclosure incorporated by reference herein.

Stress urinary incontinence is a condition characterized by involuntary loss of urine that occurs during physical activity, such as coughing, sneezing, laughing, or exercise.

Hydrocarbyl is a moiety consisting of carbon and hydrogen, including, but not limited to:
1. alkyl, which is hydrocarbyl containing no double or triple carbon-carbon bonds; alkyl includes, but is not limited to:
   linear alkyl, cyclic alkyl, branched alkyl, and combinations thereof;
   C₁₋₄ alkyl, which refers to alkyl having 1, 2, 3, or 4 carbon atoms, including, but no limited to, methyl, ethyl, isopropyl, cyclopropyl, n-propyl, n-butyl and the like;
   C₁₋₆ alkyl, which refers to alkyl having 1, 2, 3, 4, 5, or 6 carbon atoms; including, but not limited to methyl, ethyl, propyl isomers, cyclopropyl, butyl isomers, cyclobutyl, pentyl isomers, cyclopentyl, hexyl isomers, cyclohexyl, and the like;
   combinations of these terms are possible, and their meanings should be obvious to those of ordinary skill in the art; for example C₁₋₆ linear alkyl would refer to C₁₋₆ alkyl which is also linear;
2. alkenyl, which is hydrocarbyl containing one or more carbon-carbon double bonds; alkenyl includes, but is not limited to:
   linear alkenyl, cyclic alkenyl, branched alkenyl, and combinations thereof;
   alkenyl having 1, 2, 3, or more carbon-carbon double bonds;
3. alkynyl, which is hydrocarbyl containing one or more carbon-carbon triple bonds; alkynyl includes, but is not limited to:
   linear alkynyl, cyclic alkynyl, branched alkynyl, and combinations thereof;
   alkynyl having 1, 2, 3, or more carbon-carbon double bonds;
4. aryl, provided that it contains no heteroatoms either in a ring or as a substituent;
5. combinations of any of the above;
6. C₁₋₄ hydrocarbyl, which refers to hydrocarbyl having 1, 2, 3, or 4 carbon atoms; and
7. C₁₋₆ hydrocarbyl, which refers to hydrocarbyl having 1, 2, 3, 4, 5, or 6 carbon atoms.

Alkoxy is O-alkyl, such as OCH₃, O-ethyl, O-isopropyl, and the like.

Mercaptoakyl is S-alkyl, such as SCH3, S-ethyl, S-isopropyl, and the like

Acyloxy is

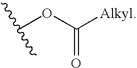

A compound, substituent, moiety, or any structural feature is stable if it is sufficiently stable for the compound to be isolated for at least 12 hours at room temperature under normal atmospheric conditions, or if it is sufficiently stable to be useful for at least one use disclosed herein.

A heavy atom is an atom which is not hydrogen.

A heteroatom is an atom which is not carbon or hydrogen.

A pharmaceutically acceptable salt is any salt that retains the activity of the parent compound and does not impart any additional deleterious or untoward effects on the subject to which it is administered and in the context in which it is administered compared to the parent compound. A pharmaceutically acceptable salt also refers to any salt which may form in vivo as a result of administration of an acid or another salt.

Pharmaceutically acceptable salts of acidic functional groups may be derived from organic or inorganic bases. The salt may comprise a mono or polyvalent ion. Of particular interest are the inorganic ions lithium, sodium, potassium, calcium, and magnesium. Organic salts may be made with amines, particularly ammonium salts such as mono-, di- and trialkyl amines or ethanol amines. Salts may also be formed with caffeine, tromethamine and similar molecules. Hydrochloric acid or some other pharmaceutically acceptable acid may form a salt with a compound that includes a basic group, such as an amine or a pyridine ring.

Unless otherwise indicated, reference to a compound should be construed broadly to include pharmaceutically acceptable salts, and tautomers of the depicted structure. For example, the structures herein are intended to include, but are not limited to, the tautomeric forms shown below.

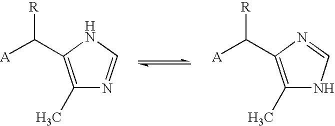

Unless stereochemistry is explicitly depicted, a structure is intended to include every possible stereoisomer, both pure or in any possible mixture.

For the purposes of this disclosure, “treat,” “treating,” or “treatment” refer to the use of a compound, composition, therapeutically active agent, or drug in the diagnosis, cure, mitigation, treatment, prevention of disease or other undesirable condition, or to affect the structure or any function of the body of man or other animals.

R is H, C₁₋₄ alkyl, or CF₃. Thus, the following compounds are contemplated.

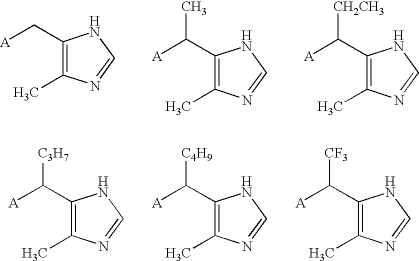

In one embodiment R is H.

A is quinolinyl having 0, 1, 2, or 3 stable substituents consisting of from 1 to 8 heavy atoms and any required hydrogen atoms, said heavy atoms being selected from C, N, O, S, F, Cl, Br, I, and any combination thereof.

Quinolinyl is one of the moieties below

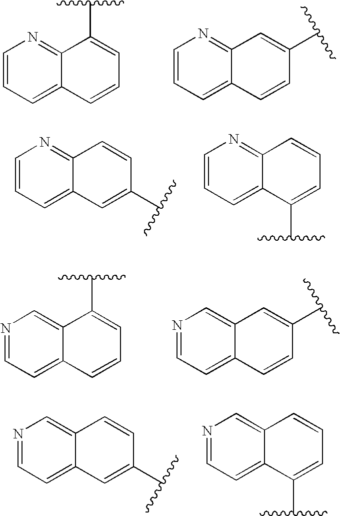

which may have substituents according to the parameters set forth herein.

Thus, for example, A may be any of the structures shown below or the like, wherein R¹, R², and R³ are independently hydrogen or stable substituents consisting of from 1 to 8 heavy atoms and any required hydrogen atoms, said heavy atoms being selected from C, N, O, S, F, Cl, Br, I, and any combination thereof; and n is 0, 1, 2, or 3.

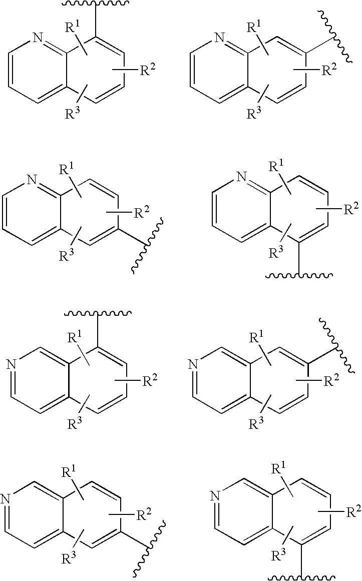

The position of R¹, R², and R³ may be anywhere on the ring system, and are not limited to the particular ring where they are located in the structural depiction.

While not intending to be limiting, examples of stable substituents consisting of from 1 to 8 heavy atoms and any required hydrogen atoms include:

hydrocarbyl, including alkyl, such as methyl, ethyl, propyl isomers, butyl isomers, and the like; alkenyl, alkynyl, and phenyl;
alkoxy,
mercaptoalkyl,
acyloxy,
amino, including NH₂, NH-alkyl, N(alkyl)₂, where the alkyl groups are the same or different;
halo, including F, Cl, Br, and I; and

### CH₂CN, CN; NO₂; OH.

If a substituent is a salt, for example of a carboxylic acid or an amine, the counterion of said salt, i.e. the ion that is not covalently bonded to the remainder of the molecule is not counted for the purposes of the number of heavy atoms in a substituent. Thus, for example, the salt —CO₂⁻Na⁺ is a stable substituent consisting of 3 heavy atoms, i.e. sodium is not counted. In another example, the salt —NH(Me)₂⁺Cl⁻ is a stable substituent consisting of 3 heavy atoms, i.e. chlorine is not counted.

In one embodiment, the substituents selected from are methyl, ethyl, propyl isomers, F, Cl, Br, I, OCH₃, NH₂, N(CH₃)₂, and combinations thereof.

In another embodiment substituents are selected from CH₃, ethyl, t-butyl, ethenyl, ethynyl, OCH₃, NHMe, NMe₂, Br, Cl, F, phenyl, and combinations thereof.

In another embodiment A is unsubstituted.

In another embodiment, the compound has the formula

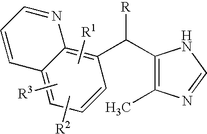

wherein R¹, R², and R³ are independently hydrogen or stable substituents consisting of from 1 to 8 heavy atoms and any required hydrogen atoms, said heavy atoms being selected from C, N, O, S, F, Cl, Br, I, and any combination thereof; and n is 0, 1, 2, or 3.

In another embodiment, the compound has the formula

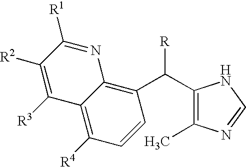

wherein R¹, R², R³, and R⁴ are independently hydrogen or stable substituents consisting of from 1 to 8 heavy atoms and any required hydrogen atoms, said heavy atoms being selected from C, N, O, S, F, Cl, Br, I, and any combination thereof; and n is 0, 1, 2, or 3.

In another embodiment, the compound has the formula

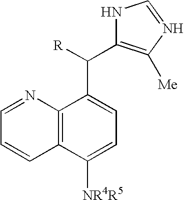

wherein R⁴ and R⁵ are independently H, C₁₋₄ alkyl, or C₁₋₅ acyl.

In another embodiment, the compound has the formula

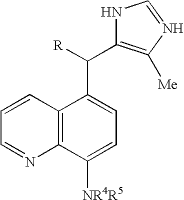

wherein R⁴ and R⁵ are independently H, C₁₋₄ alkyl, or C₁₋₅ acyl.

In another embodiment, the compound has the formula

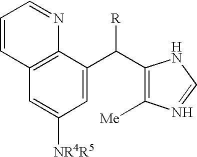

wherein R⁴ and R⁵ are independently H, C₁₋₄ alkyl, or C₁₋₅ acyl.

In another embodiment, the compound has the formula

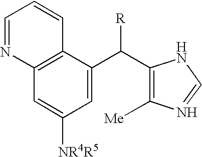

wherein R⁴ and R⁵ are independently H, C₁₋₄ alkyl, or C₁₋₅ acyl.

In another embodiment, the compound has the formula

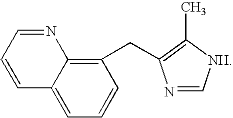

In another embodiment, the compound has the formula

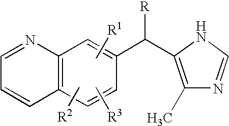

wherein R¹, R², and R³ are independently hydrogen or stable substituents consisting of from 1 to 8 heavy atoms and any required hydrogen atoms, said heavy atoms being selected from C, N, O, S, F, Cl, Br, I, and any combination thereof; and n is 0, 1, 2, or 3.

In another embodiment, the compound has the formula

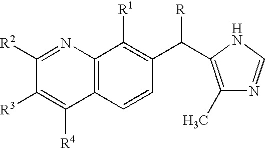

wherein R¹, R², R³, and R⁴ are independently hydrogen or stable substituents consisting of from 1 to 8 heavy atoms and any required hydrogen atoms, said heavy atoms being selected from C, N, O, S, F, Cl, Br, I, and any combination thereof; and n is 0, 1, 2, or 3.

In another embodiment, the compound has the formula

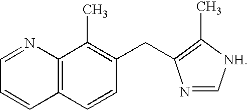

In another embodiment, the compound has the formula

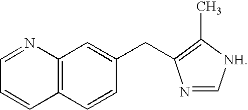

### Biological Data

### Receptor Selection and Amplification Technology (RSAT) Assay

The RSAT assay measures a receptor-mediated loss of contact inhibition that results in selective proliferation of receptor-containing cells in a mixed population of confluent cells. The increase in cell number is assessed with an appropriate transfected marker gene such as -galactosidase, the activity of which can be easily measured in a 96-well format. Receptors that activate the G protein, Gq, elicit this response. Alpha2 receptors, which normally couple to Gi, activate the RSAT response when coexpressed with a hybrid Gq protein that has a Gi receptor recognition domain, called Gq/i5.

NIH-3T3 cells are plated at a density of 2×10⁶ cells in 15 cm dishes and maintained in Dulbecco's modified Eagle's medium supplemented with 10% calf serum. One day later, cells are cotransfected by calcium phosphate precipitation with mammalian expression plasmids encoding p-SV-1-galactosidase (5-10 μg), receptor (1-2 μg) and G protein (1-2 μg). 40 μg salmon sperm DNA may also be included in the transfection mixture. Fresh media is added on the following day and 1-2 days later, cells are harvested and frozen in 50 assay aliquots. Cells are thawed and 100 μl added to 100 μl aliquots of various concentrations of drugs in triplicate in 96-well dishes. Incubations continue 72-96 hr at 37° C. After washing with phosphate-buffered saline, β-galactosidase enzyme activity is determined by adding 200 μl of the chromogenic substrate (consisting of 3.5 mM o-nitrophenyl-β-D-galactopyranoside and 0.5% nonidet P-40 in phosphate buffered saline), incubating overnight at 30° C. and measuring optical density at 420 nm. The absorbance is a measure of enzyme activity, which depends on cell number and reflects a receptor-mediated cell proliferation. The efficacy or intrinsic activity is calculated as a ratio of the maximal effect of the drug to the maximal effect of a standard full agonist for each receptor subtype. Brimonidine, also called UK14304, the chemical structure of which is shown below, is used as the standard agonist for the alpha_{2A}, alpha_{2B} and alpha_{2C} receptors. The EC₅₀ is the concentration at which the drug effect is half of its maximal effect.

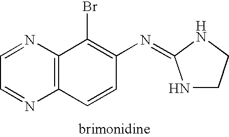

The results of the RSAT assay with several exemplary compounds of the invention are disclosed in Table 1 above together with the chemical formulas of these exemplary compounds. EC₅₀ values are nanomolar. NA stands for “not active” at concentrations less than 10 micromolar. IA stands for “intrinsic activity.”

TABLE 1

| | Alpha 1A | Alpha 2A | Alpha 2B | Alpha 2C |
|---|---|---|---|---|
| | EC50 | EC50 | EC50 | EC50 |
| Structure | (IA) | (IA) | (IA) | (IA) |
| 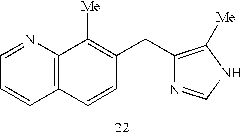 | 116 (1.10) | 284 (0.43) | 16 (1.07) | 268 (0.77) |
| 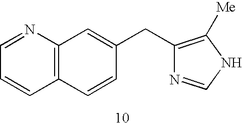 | 540 (1.05) | NA | 76 (0.96) | 521 (0.57) |
| 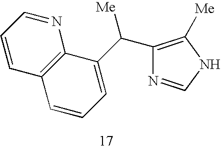 | NA | NA | 30 (0.70) | 3110 (0.32) |
| 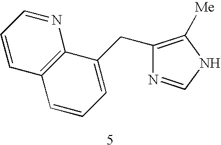 | 1730 (0.75) | NA | 117 (0.70) | NA |

Compounds 22, 10, 17, and 5 are named as follows:
8-methyl-7-((5-methyl-1H-imidazol-4-yl)methyl)quinoline (1);
7-((5-methyl-1H-imidazol-4-yl)methyl)quinoline (2);
8-(1-(5-methyl-1H-imidazol-4-yl)ethyl)quinoline (3); and
8-((5-methyl-1H-imidazol-4-yl)methyl)quinoline (4).

Compounds 5-22 are hypothetical examples of compounds that are useful as disclosed herein.

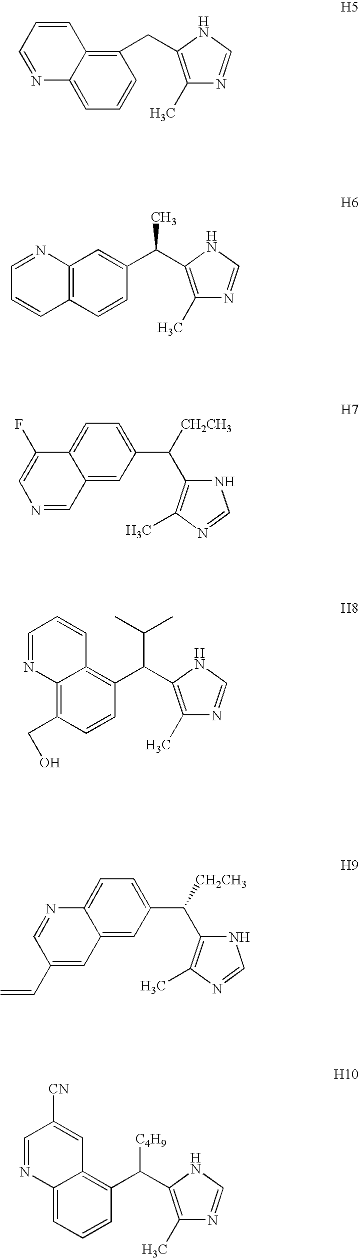

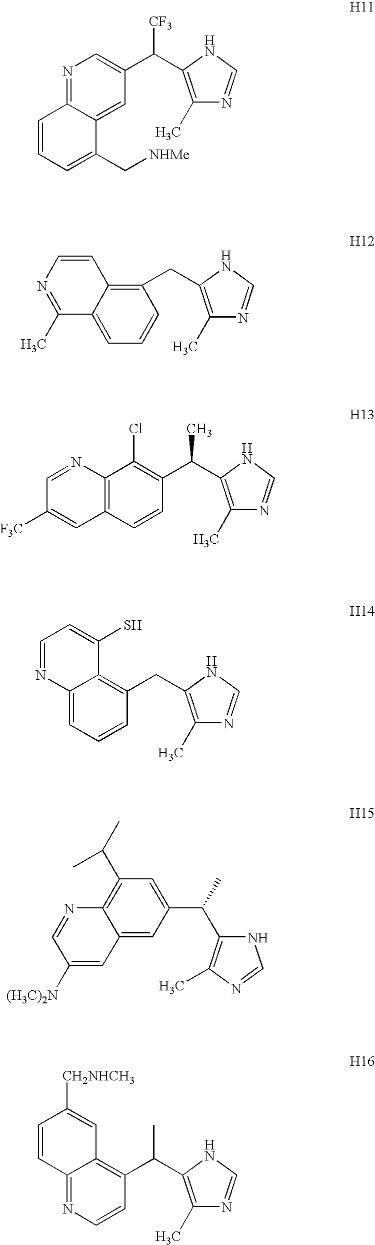

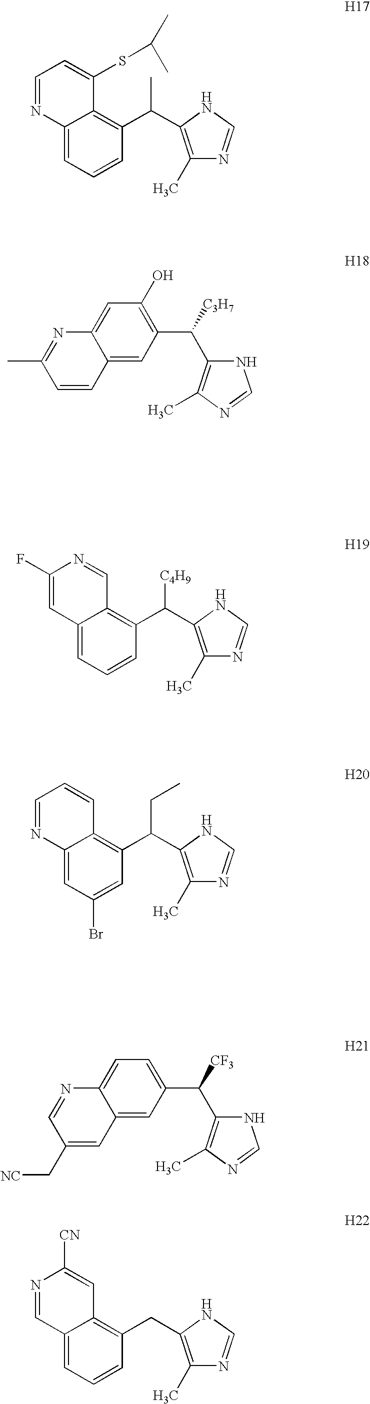

### Synthetic Methods

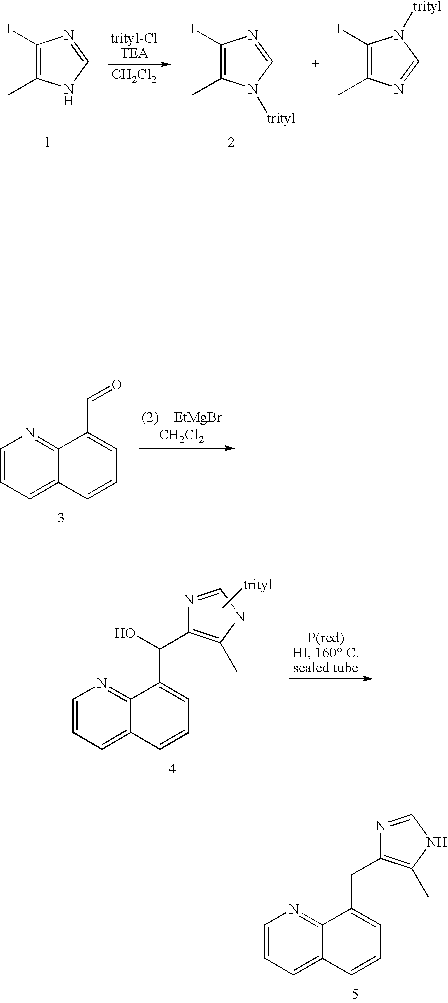

4-Iodo-5-methyl-1-trityl-1H-imidazole and 5-iodo-4-methyl-1-trityl-1H-imidazole (2): A mixture of 4-iodo-5-methyl-1H-imidazole (1) (10.5 g, 50.7 mmol) and trityl chloride (14.4 g, 50.7 mmol) in dichloromethane (100 mL) was added triethyl amine (17.6 mL, 126 mmol). The reaction mixture was stirred at room temperature (room temperature) overnight. The reaction was quenched with ammonium chloride (aq). The aqueous medium was extracted twice with dichloromethane (400 mL). The pooled organic layers were dried over magnesium sulfate. The mixture was filtered, and the solvents were removed under vacuum to give a sticky yellow solid. The crude product was triturated in hexane to give a mixture of 4-iodo-5-methyl-1-trityl-1H-imidazole and 5-iodo-4-methyl-1-trityl-1H-imidazole (2) as a white solid (20 g, 44.4 mmol, 87% yield).

(5-Methyl-1-trityl-1H-imidazol-4-yl)(quinolin-8-yl)methanol and (4-methyl-1-trityl-1H-imidazol-5-yl)(quinolin-8-yl)methanol (4): A solution of (2) (4.79 g, 10.6 mmol) in dichloromethane (70 mL was added ethyl magnesium bromide (3.0 M in diethyl ether, 3.55 mL, 10.6 mmol) dropwise at room temperature. The reaction mixture was stirred for one hour. A solution of quinoline-8-carbaldehyde (3) (1.00 g, 6.37 mmol) in dichloromethane (30 mL) was added dropwise via addition funnel. The reaction mixture was stirred at room temperature overnight. The reaction was quenched with ammonium chloride (aq). The resulting aqueous layer was extracted twice with dichloromethane (300 mL). The pooled organic layers were dried over magnesium sulfate. The mixture was filtered, and the solvents were removed under vacuum. The residue was purified by chromatography on silica gel with 100% ethyl acetate to give (5-methyl-1-trityl-1H-imidazol-4-yl)(quinolin-8-yl)methanol and (4-methyl-1-trityl-1H-imidazol-5-yl)(quinolin-8-yl)methanol (4) as a yellow foamy solid (1.40 g, 2.91 mmol, 46% yield).

8-((5-Methyl-1H-imidazol-4-yl)methyl)quinoline (5): A mixture of (4) (0.71 g, 1.48 mmol) and red phosphorus (0.46 g, 14.18 mmol) in hydroiodic acid (57% in water, 6 mL) was heated in a sealed tube at 160° C. overnight. The reaction mixture was cooled to room temperature, and the sealed tube was slowly opened to release the gas built up inside. The content was poured into crushed ice, and carefully basified with NaOH (aq) to pH >7. The aqueous layer was diluted with chloroform/isopropanol (3:1, 100 mL). The mixture was filtered through a bed of Celite to removed phosphorus. The layers were separated and the aqueous layer was extracted twice with chloroform/isopropanol (3:1, 100 mL). The pooled organic layers were dried over magnesium sulfate. The mixture was filtered and the solvents were removed under vacuum. The residue was purified by chromatography on silica gel with 2% ammonia saturated methanol in dichloromethane to give 8-((5-methyl-1H-imidazol-4-yl)methyl)quinoline (5) as a light yellow solid (0.23 g, 1.05 mmol, 71% yield). 30 mg of (5) was passed through reverse phase HPLC to give 26.5 mg of an analytically pure sample.

(5)¹H NMR (500 MHz, CDCl₃): δ 9.00 (dd, J=4.5, 2.0 Hz, 1H), 8.18 (dd, J=8.5, 1.5 Hz, 1H), 7.70 (d, J=8.0 Hz, 1H), 7.58 (d, J=7.0 Hz, 1H), 7.48-7.44 (m, 2H), 7.35 (s, 1H), 4.45 (s, 2H), 2.31 (s, 3H).

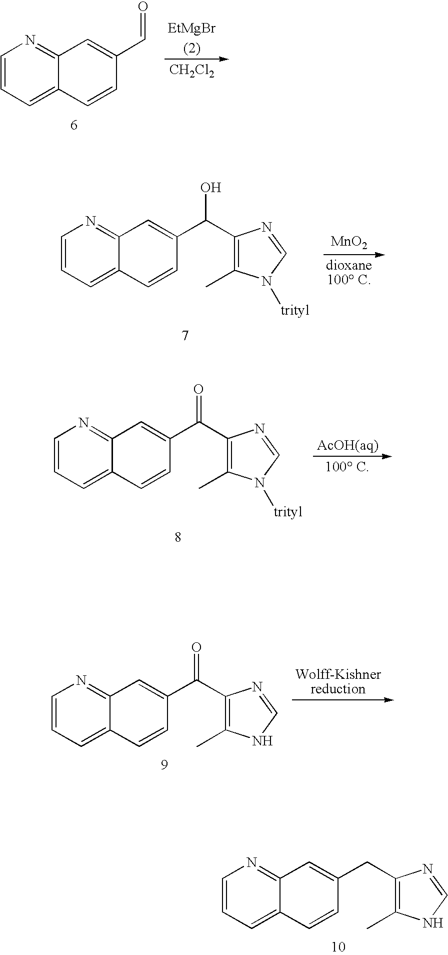

(5-Methyl-1-trityl-1H-imidazol-4-yl)(quinolin-7-yl)methanol and (4-methyl-1-trityl-1H-imidazol-5-yl)(quinolin-7-yl)methanol (7): The same procedure to make (4) was used to prepare compound (7).

(5-Methyl-1-trityl-1H-imidazol-4-yl)(quinolin-7-yl)methanone and (4-methyl-1-trityl-1H-imidazol-5-yl)(quinolin-7-yl)methanone (8): A mixture of (7) (3.45 g, 7.17 mmol), and manganese dioxide (7.33 g, 71.7 mmol) in dioxane (100 mL) was refluxed at 100° C. for 5 h. The reaction mixture was cooled to room temperature. The mixture was filtered through a bed of Celite. The filtrate was evaporated under reduced pressure. The residue was purified by chromatography on silica gel with 60% hexane and 40% ethyl acetate to afford (5-methyl-1-trityl-1H-imidazol-4-yl)(quinolin-7-yl)methanone and (4-methyl-1-trityl-1H-imidazol-5-yl)(quinolin-7-yl)methanone (8), which was carried on to the next step.

(5-Methyl-1H-imidazol-4-yl)(quinolin-7-yl)methanone (9): A solution of (8) in acetic acid/water (12 mL/8 mL) was heated at 110° C. for 1.5 h. The reaction was cooled to room temperature. Crushed ice was added, and basification of reaction with NaOH (s) to pH ˜6 was followed. The aqueous layer was extracted with chloroform/isopropanol (3:1, 200 mL). The pooled organic layers were dried over magnesium sulfate. The mixture was filtered, and the solvents were removed under vacuum. The residue was purified by chromatography on silica gel with 3% saturated ammonia methanol in dichloromethane to give (5-methyl-1H-imidazol-4-yl)(quinolin-7-yl)methanone (9) as a white solid (0.53 g, 2.24 mmol, 35% over 3 steps).

7-((5-Methyl-1H-imidazol-4-yl)methyl)quinoline (10): A mixture of (9) (0.53 g, 2.23 mmol), potassium hydroxide (0.50 g, 8.91 mol), and hydrazine hydrate (0.45 mL, 14.4 mmol) in ethylene glycol was heated at 120° C. for 1 h, then kept at 165° C. overnight. The reaction mixture was cooled to room temperature and acidified with 2 M HCl (aq) to pH ˜4. The aqueous layer was washed with chloroform/isopropanol (3:1, 200 ml). The aqueous layer was basified to pH ˜7, and extracted with chloroform/isopropanol (3:1, 200 mL). The pooled organic layers were dried over magnesium sulfate. The mixture was filtered, and the solvents were removed under vacuum. The residue was purified by chromatography on silica gel with 3% saturated ammonia methanol in dichloromethane to give 7-((5-methyl-1H-imidazol-4-yl)methyl)quinoline (10) as a yellow foam (0.32 mg, 1.45 mmol, 65% yield).

(10) ¹H NMR (500 MHz, CDCl₃): δ 8.80-8.79 (m, 1H), 8.08 (d, J=7.5 Hz, 1H), 7.78 (s, 1H), 7.68 (d, J=8.0 Hz, 1H), 7.42 (d, J=8.5 Hz, 1H), 7.38 (s, 1H), 7.31 (q, J=4.0 Hz, 1H), 4.07 (s, 2H), 2.17 (s, 3H).

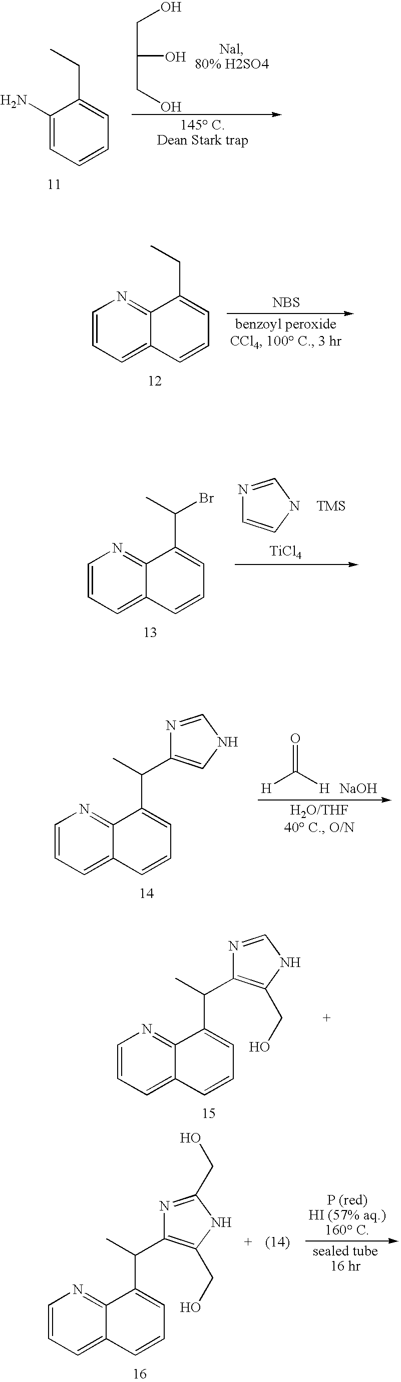

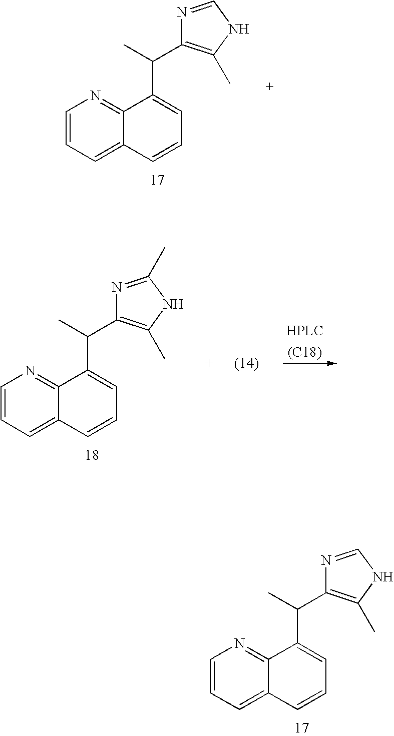

8-Ethylquinoline (12): A mixture of 2-ethylaniline (24.2 g, 200 mmol) and sodium iodide (0.40 g, 2.67 mmol) in 80% sulfuric acid (110 g) at 140° C. was added glycerine (22.0 g, 239 mmol) over a period of 30 m. The reaction mixture was stirred at 140-145° C. for 3 hours in an apparatus fitted with a Dean Stark trap. The reaction mixture was cooled to room temperature. The mixture was neutralized with 25% NaOH (aq) (210 g) to pH 7, and diluted with toluene. The mixture was extracted with ethyl acetate/ether. The pooled organic layers were washed with brine, and dried over magnesium sulfate. The mixture was filtered, and the solvents were removed under vacuum. The residue was purified by chromatography on silica gel with 5 to 7% ethyl acetate in hexane to give 8-ethylquinoline (12) (24.5 g, 156 mmol, 78% yield).

8-(1-Bromoethyl)quinoline (13): A solution of (12) (3.0 g, 19.1 mmol) in carbon tetrachloride (30 mL) was added NBS (5.10 g, 28.6 mmol), and benzoyl peroxide (0.12 g, 0.48 mmol). The mixture was heated at 100° C. for 3 hours. The reaction was cooled to room temperature. The mixture was filtered through filter paper, and washed with ethyl acetate. The filtrate was adsorbed into silica gel. The mixture was purified by chromatography on silica gel with 5 to 15% ethyl acetate in hexane to give 8-(1-bromoethyl)quinoline (13) (3.5 g, 14.8 mmol, 78% yield).

8-(1-(1H-Imidazol-4-yl)ethyl)quinoline (14): Titanium tetrachloride (3.28 ml, 29.9 mmol) was added to anhydrous chloroform (25 mL) at 0° C. 1-Trimethylsilanyl-1H-imidazole (4.38 ml, 29.9 mmol) in chloroform (25 ml) was added slowly (6 to 7 m) to the TiCl₄ solution. The resulting orange solid mixture was stirred at 0° C. for 30 m followed by the addition of (13) (3.53 g, 15.0 mmol) in chloroform (15 mL). The mixture was warmed to room temperature and stirred overnight. The mixture was quenched with water (60 mL). The two layers were separated and the organic layer was extracted twice with water (40 mL). The pooled aqueous layer was neutralized with 4 M NaOH to pH >8. The basic aqueous layer was extracted with dichloromethane numerous times. The pooled organic layers were washed with brine once, and dried over magnesium sulfate. The mixture was filtered, and the solvent was removed under vacuum. The residue was purified by chromatography on silica gel with 2 to 5% saturated ammonia methanol in dichloromethane to give 8-(1-(1H-imidazol-4-yl)ethyl)quinoline (14) as an off white solid (1.61 g, 7.22 mmol, 48% yield.)

(4-(1-(Quinolin-8-yl)ethyl)-1H-imidazol-5-yl)methanol (15): A solution of (14) (0.41 g, 1.84 mmol) in THF/H₂O (4 mL/2 mL) was added 2 N NaOH (1.90 mL, 3.80 mmol), and formaldehyde (aq) (37%, 0.14 mL, 1.88 mmol). The reaction mixture was stirred at 40° C. overnight. TLC and mass spectrometry analyses showed starting material (14), (4-(1-(quinolin-8-yl)ethyl)-1H-imidazol-5-yl)methanol (15), and (4-(1-(quinolin-8-yl)ethyl)-1H-imidazole-2,5-diyl)dimethanol (16). The solvent was evaporated under reduce pressure and the residue was carried on to the next step without further purification.

8-(1-(5-Methyl-1H-imidazol-4-yl)ethyl)quinoline (17): The same synthetic method to make (5) was used. The crude product consisted of (14), 8-(1-(5-methyl-1H-imidazol-4-yl)ethyl)quinoline (17), and 8-(1-(2,5-dimethyl-1H-imidazol-4-yl)ethyl)quinoline (18). The mixture was purified by reverse phase HPLC to give (17) as a solid (0.077 g, 0.32 mmol, 18% over 2 steps).

(17) ¹H NMR (500 MHz, CDCl₃): δ 8.96 (dd, J=4.5, 2.0, 1H), 8.14 (dd, J=8.5, 1.5 Hz, 1H), 7.65 (d, J=7.5 Hz, 1H), 7.46-7.38 (m, 2H), 7.41 (s, 1H), 5.23 (bs, 1H), 2.22 (s, 3H), 1.81 (d, J=7.5 Hz, 3H).

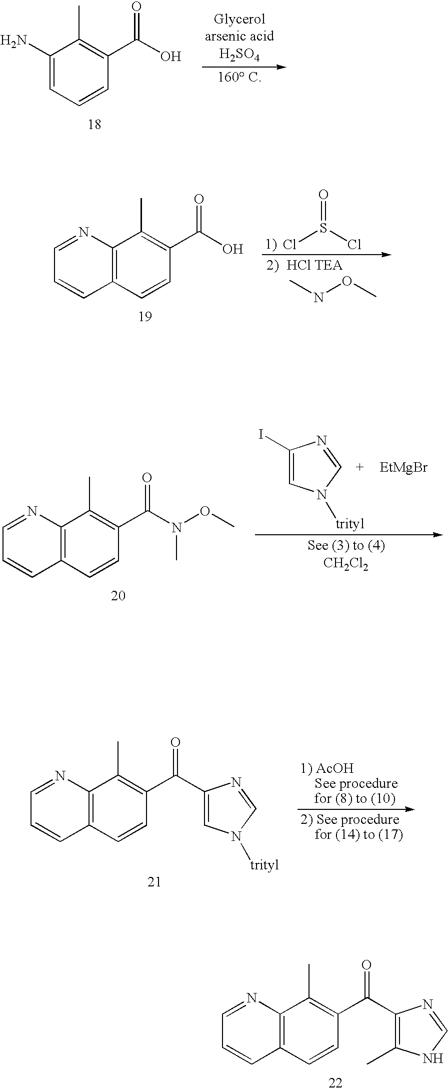

8-Methylquinoline-7-carboxylic acid (19): A mixture of 3-amino-2-methylbenzoic acid (18) (6.1 g, 39.7 mmol), arsenic acid (7.4 g, 52.3 mmol), and glycerol (5.8 mL, 79.4 mmol) in sulfuric acid (9 mL) was heated at 160° C. for 5 hours. The reaction mixture was cooled to room temperature and diluted with water. The mixture was filtered through a bed of celite and the filtrate was adjusted with 2 M NaOH to pH ˜6. The aqueous layer was extracted numerous times with chloroform/isopropanol. The pooled organic layers were removed under vacuum. The solid residue was triturated with chloroform. The mixture was filtered, and the solid was washed with hexane and dried under high vacuum to give 8-methylquinoline-7-carboxylic acid (19) 3.86 g (20.6 mmol, 52% yield).

N-Methoxy-N,8-dimethylquinoline-7-carboxamide (20): (19) (3.86 g, 20.6 mmol) was refluxed in thionyl chloride (15 mL, 204 mmol) for one hour. The reaction mixture was cooled to room temperature and the thionyl chloride was removed under vacuum. The residue was diluted with dichloromethane and the solvent was removed under vacuum. The solid residue was solvated with dichloromethane (120 mL), N,O-dimethylhydroxylamine hydrochloride (3.0 g, 30.1 mmol), and triethylamine (10.6 mL, 76.0 mmol) at 0° C., and the mixture was stirred for several hours. The reaction mixture was quenched with water, and extracted with dichloromethane. The pooled organic layers were dried over magnesium sulfate. The mixture was filtered, and the solvents were removed under vacuum to give the crude product as an oil. The oil was purified by chromatography on silica gel with 50% hexane:ethyl acetate to 40% hexane:ethyl acetate to give N-methoxy-N,8-dimethylquinoline-7-carboxamide (20) as a yellow oil (3.9 g, 17.0 mmol, 82% yield).

(8-Methylquinolin-7-yl)(1-trityl-1H-imidazol-4-yl)methanone (21) was synthesized from (20) and 4-iodo-1-trityl-1H-imidazole via the procedure used in the preparation of (4) from (3) above.

8-Methyl-7-((5-methyl-1H-imidazol-4-yl)methyl)quinoline (22): (21) was subjected to the conditions above for steps 8 to 10, and 14 to 17 to yield (22).

8-Methyl-7-((5-methyl-1H-imidazol-4-yl)methyl)quinoline (22): ¹H NMR (500 MHz, CD₃OD): δ 8.84 (dd, J=4.5, 1.5 Hz, 1H), 8.23 (dd, J=8.5, 1.5 Hz, 1H), 7.67 (d, J=8.0 Hz, 1H), 7.49 (s, 1H), 7.44 (dd, J=8.5, 2.0 Hz, 1H), 7.34 (d, J=8.0 Hz, 1H), 4.15 (s, 2H), 2.79 (s, 3H), 2.14 (s, 3H).

Additional substitution on the quinolinyl ring of A may be obtained by purchasing the corresponding substituted quinolinecarbaldehyde, e.g. substituted versions of 3 or 6; or by purchasing substituted anilines, e.g. substituted versions of 11 or 18. Alternatively, additional substituents may be added to the quinolinyl ring by methods known in the art.

Different R groups may be obtained by using the appropriate analog of 11 or treating 21 or an analog with RMgBr or an equivalent reagent.

Other alternate routes to a wide variety of compounds are readily apparent to those skilled in the art.

These compounds will be useful for the treatment of mammals, including humans, with a range of conditions and diseases that include, but are not limited to, ischemic neuropathies, optic neuropathy, neuropathic pain, visceral pain, corneal pain, headache pain, migraine, cancer pain, back pain, irritable bowel syndrome pain, muscle pain and pain associated with diabetic neuropathy, the treatment of diabetic retinopathy, other retinal degenerative conditions, cognitive deficits, neuropsychiatric conditions, drug dependence and addiction, withdrawal symptoms, spasticity, autism, Huntington's disease, attention deficit disorder, attention deficit hyperactivity disorder ADHD, obsessive-compulsive disorders, Tourette's disorder, Parkinson's ALS, and other motor or movement disorders and diseases.

Other uses include dilation of the pupil, increase blood pressure, treating nasal congestion, and vasoconstriction in ocular tissue.

These compounds may be formulated into solid, liquid, or other types of dosage forms using methods known in the art. Both formulation of dosage forms and determination of a therapeutically effective dose can be readily made by a person of ordinary skill using routine methods.

The foregoing description details specific methods and compositions that can be employed to practice the present invention, and represents the best mode contemplated. However, it is apparent for one of ordinary skill in the art that further compounds with the desired pharmacological properties can be prepared in an analogous manner, and that the disclosed compounds can also be obtained from different starting compounds via different chemical reactions. Similarly, different pharmaceutical compositions may be prepared and used with substantially the same result. Thus, however detailed the foregoing may appear in text, it should not be construed as limiting the overall scope hereof; rather, the ambit of the present invention is to be governed only by the lawful construction of the claims.

What is claimed is:

1. A method for treating a disorder associated with selective subtype modulation of alpha 2B and alpha 2C adrenergic receptors comprising administering to a subject in need thereof a pharmaceutical composition containing a therapeutically effective amount of at least one compound having the structure: 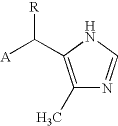
wherein R is H, C₁₋₄ alkyl, or CF₃;
A is quinolinyl having 0, 1, 2, or 3 stable substituents consisting of from 1 to 8 heavy atoms and any required hydrogen atoms, said heavy atoms being selected from C, N, O, S, F, Cl, Br, I, and any combination thereof.

2. The compound of claim 1 wherein R is H.

3. The compound of claim 1 wherein said substituents are selected from CH₃, ethyl, t-butyl, ethenyl, ethynyl, OCH₃, NHMe, NMe₂, Br, Cl, F, phenyl, and combinations thereof.

4. The method of claim 1 wherein A is unsubstituted.

5. The method of claim 1, wherein said compound is further characterized by the formula: 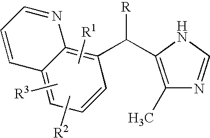
wherein R¹, R², and R³ are independently hydrogen or stable substituents consisting of from 1 to 8 heavy atoms and any required hydrogen atoms, said heavy atoms being selected from C, N, O, S, F, Cl, Br, I, and any combination thereof; and n is 0, 1, 2, or 3.

6. The method of claim 5, wherein said compound is further characterized by the formula:

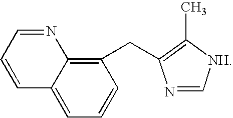

7. The method of claim 1, wherein said compound is further characterized by the formula: 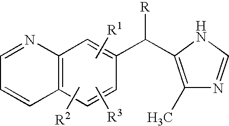
wherein R¹, R², and R³ are independently hydrogen or stable substituents consisting of from 1 to 8 heavy atoms and any required hydrogen atoms, said heavy atoms being selected from C, N, O, S, F, Cl, Br, I, and any combination thereof; and n is 0, 1, 2, or 3.

8. The method of claim 7, wherein said compound is further characterized by the formula:

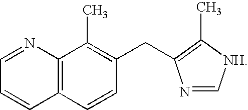

8. The method of claim 7, wherein said compound is further characterized by the formula:

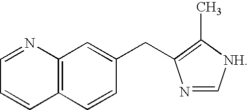

9. The method of claim 3, wherein said compound is selected from:
8-methyl-7-((5-methyl-1H-imidazol-4-yl)methyl)quinoline;
7-((5-methyl-1H-imidazol-4-yl)methyl)quinoline;
8-(1-(5-methyl-1H-imidazol-4-yl)ethyl)quinolin; and
8-((5-methyl-1H-imidazol-4-yl)methyl)quinoline.

10. The method according to any one of claims 1 R is methyl, ethyl, or CF₃.

11. The method of claim 1, wherein said compound is further characterized by the formula: 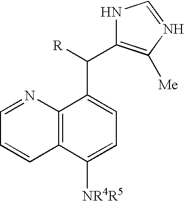
wherein R⁴ and R⁵ are independently H, C₁₋₄ alkyl, or C₁₋₅ acyl.

12. The method of claim 1, wherein said compound is further characterized by the formula: 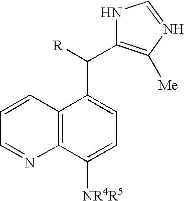
wherein R⁴ and R⁵ are independently H, C₁₋₄ alkyl, or C₁₋₅ acyl.

13. The method of claim 1, wherein said compound is further characterized by the formula: 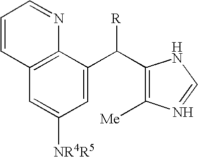
wherein R⁴ and R⁵ are independently H, C₁₋₄ alkyl, or C₁₋₅ acyl.

14. The method of claim 1, wherein said compound is further characterized by the formula: 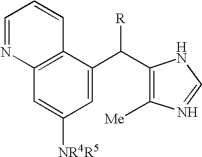
wherein R⁴ and R⁵ are independently H, C₁₋₄ alkyl, or C₁₋₅ acyl.

15. The method of claim 1 wherein the disorder is an ocular disorder.

16. The method of claim 15 wherein the ocular disorder is glaucoma, elevated intraocular pressure, optic neuropathy, corneal pain, diabetic retinopathy, retinal dystrophies, macular degeneration, non-exudative age related macular degeneration (ARMD), exudative Age Related Macular Degeneration (ARMD), Lebers optic neuropathy, optic neuritis often associated with multiple sclerosis, retinal vein occlusions, ischemic neuropathies and other neurodegenerative diseases, choroidal neovascularization, central serous chorioretinopathy, cystoid macular edema, diabetic macular edema, myopic retinal degeneration, acute multifocal placoid pigment epitheliopathy, Behcet's disease, birdshot retinochoroidopathy, intermediate uveitis (pars planitis), multifocal choroiditis, multiple evanescent white dot syndrome (MEWDS), ocular sarcoidosis, posterior scleritis, serpiginous choroiditis, subretinal fibrosis and uveitis syndrome, Vogt-Koyanagi-Harada syndrome, punctate inner choroidopathy, acute posterior multifocal placoid pigment epitheliopathy, acute retinal pigment epithelitis, acute macular neuroretinopathy and disorders following procedures such as photodynamic therapy and laser-assisted in situ keratomileusis (LASIK).

17. The method of claim 1 wherein the disorder is chronic pain, visceral pain, neuropathic pain, cancer pain, post-operative pain, allodynic pain, neuropathic pain, causalgia, ischemic neuropathies, neurodegenerative diseases, diarrhea, nasal congestion, muscle spasticity, diuresis, withdrawal syndromes, neurodegenerative diseases, optic neuropathy, spinal ischemia, stroke, memory and cognition deficits, attention deficit disorder, psychoses, manic disorders, anxiety, depression, hypertension, congestive heart failure, cardiac ischemia, arthritis, spondylitis, gouty arthritis, osteoarthritis, juvenile arthritis, autoimmune diseases, lupus erythematosus, chronic gastrointestinal inflammations, Crohn's disease, gastritis, irritable bowel syndrome (IBS), functional dyspepsia, ulcerative colitis, allodynia, or a combination thereof.

18. The method of claim 17, wherein the disorder is chronic pain, neuropathic pain, or visceral pain.

19. The method of claim 1 wherein the disorder is a central nervous system (CNS) motor disorder.

20. The method of claim 19 wherein the disorder is L-dopa-induced dyskinesias, tardive dyskinesias, cervical dystonia, spinal torticollis, blepharospasm/Meige's disease, restless leg syndrome, essential tremor, rigidity (Parkinson's disease-associated or otherwise specified), ataxic disorder, or spasticity.

21. A method for treating a disorder associated with modulation of alpha 1A adrenergic receptors comprising administering to a subject in need thereof a pharmaceutical composition containing a therapeutically effective amount of at least one compound having the structure: 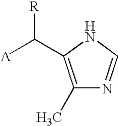
wherein R is H, C₁₋₄ alkyl, or CF₃;
A is quinolinyl having 0, 1, 2, or 3 stable substituents consisting of from 1 to 8 heavy atoms and any required hydrogen atoms, said heavy atoms being selected from C, N, O, S, F, Cl, Br, I, and any combination thereof.

22. The method of claim 21 wherein the disorder is stress urinary incontinence as well as other uses including dilation of the pupil, increase blood pressure, treating nasal congestion, or vasoconstriction in ocular tissue.
